(19) United States
(12) Patent Application Publication
(10) Pub. No.: US 2004/0013635 A1
(43) Pub. Date: Jan. 22, 2004

(54) Method of treating skin with soluble protein fractions

(76) Inventors: Christine Jeanmaire, Nancy (FR); Marc Pauly, Chateau-Salins (FR); Louis Danoux, Sauixures-les-Nancy (FR)

Correspondence Address: COGNIS CORPORATION, PATENT DEPARTMENT, 300 BROOKSIDE AVENUE, AMBLER, PA, 19002, US

(21) Appl. No.: 10/333,438

(22) PCT Filed: Jul. 10, 2001
(86) PCT No.: PCT/EP01/07946

(30) Foreign Application Priority Data
Jul. 19, 2000 (FR) 00/09486

(51) Int. Cl. A61K 7/06; A61K 35/78

(52) U.S. Cl. 424/74; 424/769; 424/757

(57) ABSTRACT

Methods of treating skin and for stimulating filaggrin synthesis are described wherein skin is contacted with a soluble protein fraction, preferably extracted from leguminous seeds, and wherein the fraction (i) exhibits at least one band under non-reducing conditions in polyacrylamide gel electrophoresis with sodium dodecyl sulfate, (ii) has an average molecular weight of 500 to 500,000 dalton, (iii) has a total nitrogen content of from 0.005 to 0.5% by weight based on the percentage protein content and an amino nitrogen content of from 0.0005 to 0.01% by weight; and (iv) wherein the fraction is soluble in water and aqueous electrolyte solutions and insoluble in ethanol or acetone, and (v) wherein the fraction forms deposits in aqueous solution with a reactant selected from the group consisting of trichloroacetic acid and picric acid.

### FIELD OF THE INVENTION

This invention relates generally to cosmetics and, more particularly, to the use of special extracts for stimulating the synthesis of proteins characteristic of the differentiation of keratinocytes, more particularly for stimulating the synthesis of filaggrin.

### PRIOR ART

Profilaggrin is the principal constituent of the keratohyalin granules in the cells of the granular layer of the tissue epidermis. The profilaggrin synthesized and phosphorylated in the Stratum granulosum is a protein with a molecular weight of ca. 400 kDa and acts as a precursor in the biosynthesis of filaggrin into which it is converted during the maturation of the keratinocytes by dephosphorylation and proteolysis. Human filaggrin has a molecular weight of ca. 37 kDa, is cationically charged and is usually to be found in the Stratum corneum of the tissue epidermis [cf. Sarret et al., Path. Biol. 37(4), 297 (1989)].

It is known that filaggrin plays an important part in the aggregation of keratin in the lower Stratum corneum. The degradation products of filaggrin, namely free amino acids and derivatives thereof, prevent the loss of water from the Stratum corneum. Filaggrin is thus an essential reservoir for natural moisturizing factors (NMFs). It has been shown that a reduction in the concentration of filaggrin, which can be caused in particular by aging, is accompanied by the occurrence of dry skin [cf. T. Tezuka et al., Dermatology, 1994, 188, 21-24] and wrinkling [cf. J. I. Contet-Audonneau et al., British J. Dermatology, 1999, 140, 1038-1047].

Accordingly, the problem addressed by the present invention was to find new active substances with which the synthesis of proteins characteristic of the differentiation of keratinocytes, more particularly the synthesis of filaggrin, could be stimulated in order to counteract in particular ageing of the skin and drying out of the skin.

### DESCRIPTION OF THE INVENTION

The present invention relates to the use of extracted soluble protein fractions which

(a) show at least one band under non-reducing conditions in polyacrylamide gel electrophoresis with sodium dodecyl sulfate;

(b) have an average molecular weight of 500 to 500,000 and preferably 5,000 to 100,000 dalton;

(c) have a total nitrogen content, based on the percentage protein content, of 0.005 to 0.5% by weight and an amino nitrogen content of 0.0005 to 0.01% by weight;

(d) are soluble in water and aqueous electrolyte solutions, but insoluble in ethanol or acetone; and

(e) form deposits in aqueous solutions together with trichloroacetic acid or picric acid,

for stimulating the synthesis of proteins characteristic of the differentiation of keratinocytes, more particularly for stimulating the synthesis of filaggrin.

It has surprisingly been found that the administration of protein fractions which meet the conditions mentioned above and which are obtained in particular from the seeds of the bambara nut stimulate the synthesis of filaggrin and thus counteract the effect of drying out and wrinkling.

Soluble Protein Extracts

According to the invention, fractions obtained by extraction of leguminous seeds, more particularly by extraction of seeds of the bambara nut, are preferably used. The bambara nut (Voandzeia subterranea (L) Thouars) is a seed of African origin eaten locally as a vegetable. It is an indigenous African vegetable grown mainly by farmers as a “famine crop”, its most important characteristic being its tolerance to drought and poor soils and its ability to grow under conditions unsuitable for peanuts. Bambara nut seeds, which represent a complete food, contain proteins, carbohydrates and lipids and can be eaten at different stages of ripeness. Their chemical composition (g/100 g flour or 100 g dried seeds) is as follows:

| | |
|---|---|
| proteins: | 16 to 21% by weight, |
| starch: | 39 to 49.5% by weight, |
| tannins | (expressed as tannic acid): 0.36 to 0.94% by weight, |
| lipids: | 5 to 7.3% by weight, |
| ash: | 3.65% by weight. |

It is known that the seed of Voandzeia subterranea contains protease inhibitors and the trypsin-inhibiting activity as estimated by the so-called Kakadé technique is—according to the literature—from 6.7 to 15.4 TIU/mg flour, the functional properties of the protein isolates of this seed having been investigated for food purposes. Reference is made in this connection to International patent application WO 98/42305 from which the use of bambara nut extracts in cosmetics is known. The document in question also contains further information on the production of the extracts. In addition, it has proved to be of advantage to use fractions which contain at least one protease inhibitor.

The extracted soluble protein fractions according to the invention are preferably used

against ageing of the skin and particularly against wrinkling,

for the treatment of dry skin and/or

against the drying out of the skin.

According to the invention, the extracted soluble protein fractions according to the invention are active against ageing of the skin and more particularly against any form of lining and wrinkling. Another name for care preparations of this type is anti-ageing preparations. The uses include slowing down of skin ageing processes. The protein fractions according to the invention are also active against drying out of the skin because, by stimulating the synthesis of proteins for differentiating keratinocytes, more particularly for stimulating the synthesis of filaggrin, the proportion of these proteins in the keratinocytes of the Stratum corneum is increased. Their degradation products, the free amino acids and derivatives thereof, prevent the skin from drying out. Filaggrin in particular is an essential reservoir for natural moisturizing factors (NMFs). Besides preventing the skin from drying out, the protein fractions according to the invention may also be used to treat dry skin and to give back at least the natural moisture content.

### EXAMPLES

### Production Example 1

250 g of ground Voandzeia subterranea seeds were dispersed in 2.5 l of distilled water. After stirring for 15 minutes, the pH was adjusted to 7.5 by addition of sodium hydroxide and extraction was carried out for 1 h at room temperature. After centrifuging (10 mins., 5,000 G), the upper oily phase was discarded and the yellowish aqueous phase was purified by ultrafiltration (retention at 15,000 Da, concentration factor 2 to 10) or diafiltration with water. The fraction obtained in this way had a dry residue of 1 to 3% by weight and a protein concentration of 0.3 to 15 g/l (biuret determination). The fraction was then dried by freeze drying. The anti-trypsic activity of the dried product amounted to 50-200 TUI/mg (as determined by the Kakadé method) or, based on the dry residue of the solution, to 800-2,500 TUI/ml.

### Production Example 2

400 g of ground Voandzeia subterranea seeds were dispersed in 4 l of distilled water and extracted as described in Example 1. 3.2 l of a substantially colorless solution with a dry residue of 3% by weight and a protein concentration of 0.3 to 15 g/l were obtained. The pH of the solution was adjusted to 4.5 by addition of sulfuric acid, followed by stirring for 30 mins. After centrifuging (15 mins., 5,000 G), the upper oily phase was discarded and the yellowish aqueous phase was purified by ultrafiltration (retention at 15,000 Da, concentration factor 2 to 10) or diafiltration with water. The fraction obtained in this way had a dry residue of 1 to 3% by weight and a protein concentration of 0.3 to 15 g/l (biuret determination). The fraction was then dried by freeze drying. The anti-trypsic activity of the dried product amounted to 50-200 TUI/mg (as determined by the Kakadé method) or, based on the dry residue of the solution, to 800-2,500 TUI/mI.

### Production Example 3

The extract obtained in accordance with Example 1 was subjected to gel permeation in a Superose 12 HR FPLC column (manufacturer:Pharmacia). Five fractions were obtained:

| | |
|---|---|
| fraction 1: | molecular weight >500,000 Da |
| fraction 2: | molecular weight 100,000 to 500,000 Da |
| fraction 3: | molecular weight 30,000 to 100,000 Da |
| fraction 4: | molecular weight 5,000 to 30,000 Da |
| fraction 5: | molecular weight <5,000 Da |

Cell Growth Test.

The following activity tests were carried out using, on the one hand, an extract according to Example 2 and, on the other hand, the commercially available formulation Filladyn® (Laboratoires Sérobiologiques S.A.) which contained 60% of the extract of Example 2 and, in addition, polyols, xanthan gum and buffer salts.

The influence of the preparations on human fibroblasts, with which the regeneration capacity of the cells was to be tested, was investigated as follows:

A standard cell medium (DMEM) containing 10% fetal calf serum (FCS) was inoculated with human fibroblasts. After incubation for 1 day at 37°/5% CO₂ concentration, the growth medium was replaced by one with no FCS which contained the test preparations in concentrations of 0.03 to 0.6% by volume. After incubation for 3 days, cell growth was determined by determination of the cellular protein content (Bradford method) and the ATP essential for phosphorylation of the profilaggrin (Vasseur method). The results are set out in Table 1 where they are expressed as the relative percentage content, based on a standard with none of the test substances added (=100%).

TABLE 1
Cell growth

Bambara nut extract

| Test concentration (% by volume) | Proteins | ATP |
|---|---|---|
| 0 | 100 | 100 |
| 0.03 | 111 | 100 |
| 0.1 | 121 | 111 |
| 0.3 | 143 | 114 |
| 0.6 | 171 | 157 |

Stimulation of Filaggrin Synthesis (in vitro).

A standard cell medium (DMEM) containing 10% fetal calf serum (FCS) was inoculated with human keratinocytes. After incubation for 4 days at 37° C./5% CO₂ concentration, the growth medium was replaced by one with no FCS which contained the test preparations in concentrations of 0.025 to 2% by volume. After incubation for 6 to 7 days, the synthesis of profilaggrin/filaggrin was determined immunohistochemically, i.e. using a monoclonal antibody that recognizes profilaggrin or filaggrin. The results are set out in Table 2.

Stimulation of Profilaggrin/filaggrin Synthesis (Ex Vivo).

Human biopsies from plastic surgery were disinfected and cultures were prepared (DMEM, 37° C.). The test substances (0.3% by weight bambara nut extract and 5% by weight Filladyn®) incorporated in a hydrogel were topically applied (5 treatments in 3 days). After 3 days, narrow biopsies were prepared and frozen in liquid nitrogen. The formation of profilaggrin/filaggrin was then investigated—again by the immunohistochemical method. The results are set out in Table 3.

The profilaggrin/filaggrin was then microscopically determined with a confocal laser scanning microscope. The micrographs were converted into numerical values using Leica's Quantimet Q500/W software and analyzed. The results are expressed as the percentage surface area covered by profilaggrin/filaggrin in the histological sections.

TABLE 2
Profilaggrin/filaggrin synthesis

| Test concentration | Bambara nut extract | | | Filladyn ® | | |
|---|---|---|---|---|---|---|
| (% by volume) | t = 0 d | t = 6 d | t = 7 d | t = 0 d | t = 6 d | t = 7 d |
| 0 | 0.7 ± 0.1 | 7 ± 0.9 | 7 ± 0.6 | | | |
| 0.025 | 0.7 ± 0.1 | 22 ± 1.3 | 28 ± 1.7 | | | |
| 0.1 | 0.7 ± 0.1 | 34 ± 2.1 | 34 ± 1.0 | | | |
| 2 | | | | 0.7 ± 0.1 | 20 ± 1.4 | 33 ± 1.3 |

TABLE 3
Profilaggrin/filaggrin synthesis

| Control | Placebo hydrogel | Bambara nut extract | Filladyn® |
|---|---|---|---|
| 16 ± 1.5 | 18 ± 0.5 | 22 ± 1.2 | 21 ± 1.6 |

Some Formulation Examples are set out Table 4 below.

Table 4
Examples of cosmetic preparations
(water, preservative to 100% by weight)

| Composition (INCI) | 1 | 2 | 3 | 4 | 5 |
|---|---|---|---|---|---|
| Emulgade ® SE | 5.0 | 5.0 | 4.0 | — | — |
| Glyceryl Stearate (and) | | | | | |
| Ceteareth 12/20 (and) Cetearyl | | | | | |
| Alcohol (and) Cetyl Palmitate | | | | | |
| Eumulgin ® B1 | — | — | 1.0 | — | — |
| Ceteareth-12 | | | | | |
| Lameform ® TGI | — | — | — | 4.0 | — |
| Polyglyceryl-3 Isostearate | | | | | |
| Dehymuls ® PGPH | — | — | — | — | 4.0 |
| Polyglyceryl-2 Dipolyhydroxystearate | | | | | |
| Monomuls ® 90-O 18 | — | — | — | 2.0 | — |
| Glyceryl Oleate | | | | | |
| Cetiol ® HE | — | — | — | — | 2.0 |
| PEG-7 Glyceryl Cocoate | | | | | |
| Cetiol ® OE | — | — | — | 5.0 | 6.0 |
| Dicaprylyl Ether | | | | | |
| Cetiol ® PGL | — | — | 3.0 | 10.0 | 9.0 |
| Hexyldecanol (and) Hexyldecyl | | | | | |
| Laurate | | | | | |
| Cetiol ® SN | 3.0 | 3.0 | — | — | — |
| Cetearyl Isononanoate | | | | | |
| Cetiol ® V | 3.0 | 3.0 | — | — | — |
| Decyl Oleate | | | | | |
| Myritol ® 318 | — | — | 3.0 | 5.0 | 5.0 |
| Coco Caprylate Caprate | | | | | |
| Bees Wax | — | — | — | | |
| Nutrilan ® Elastin E20 | 2.0 | — | — | — | — |
| Nutrilan ® I-50 | — | 2.0 | — | — | — |
| Hydrolyzed Collagen | | | | | |
| Gluadin ® AGP | — | — | 0.5 | — | — |
| Hydrolyzed Wheat Gluten | | | | | |
| Gluadin ® WK | — | — | — | 0.5 | 0.5 |
| Sodium Cocoyl Hydrolyzed | | | | | |
| Wheat Protein | | | | | |
| Filladyn @ | 1.0 | 1.0 | 1.0 | 1.0 | 1.0 |
| Hydagen ® CMF | 1.0 | 1.0 | 1.0 | 1.0 | 1.0 |
| Chitosan | | | | | |
| Magnesium Sulfate Hepta Hydrate | — | — | — | 1.0 | 1.0 |
| Glycerin (86% by weight) | 3.0 | 3.0 | 5.0 | 5.0 | 3.0 |

(1) Soft cream, (2,3) moisturizing emulsion, (4,5) night cream

The registered trade marks and brand names shown in Table 4 are products of the Cognis Group.

1. The use of extracted soluble protein fractions which
(a) show at least one band under non-reducing conditions in polyacrylamide gel electrophoresis with sodium dodecyl sulfate;
(b) have an average molecular weight of 500 to 500,000 dalton;
(c) have a total nitrogen content, based on the percentage protein content, of 0.005 to 0.5% by weight and an amino nitrogen content of 0.0005 to 0.01% by weight;
(d) are soluble in water and aqueous electrolyte solutions, but insoluble in ethanol or acetone; and
(e) form deposits in aqueous solutions together with trichloroacetic acid or picric acid,
for stimulating the synthesis of proteins characteristic of the differentiation of keratinocytes, more particularly for stimulating the synthesis of filaggrin.

2. The use claimed in claim 1, characterized in that fractions containing at least one protein inhibitor are used.

3. The use claimed in claims 1 and/or 2, characterized in that fractions obtained by extracting leguminous seeds are used.

4. The use claimed in claim 3, characterized in that fractions obtained by extracting seeds of the bambara nut (Voandzeia subterranea) are used.

5. The use of extracted soluble protein fractions as claimed in any of claims 1 to 4 against ageing of the skin, more particularly against wrinkling.

6. The use of extracted soluble protein fractions as claimed in any of claims 1 to 4 for treating dry skin and/or against drying out of the skin.

7. A method of stimulating filaggrin synthesis, said method comprising:
(a) providing a soluble protein fraction, wherein the fraction (i) exhibits at least one band under non-reducing conditions in polyacrylamide gel electrophoresis with sodium dodecyl sulfate, (ii) has an average molecular weight of 500 to 500,000 dalton, (iii) has a total nitrogen content of from 0.005 to 0.5% by weight based on the percentage protein content and an amino nitrogen content of from 0.0005 to 0.01% by weight; and (iv) wherein the fraction is soluble in water and aqueous electrolyte solutions and insoluble in ethanol or acetone, and (v) wherein the fraction forms deposits in aqueous solution with a reactant selected from the group consisting of trichloroacetic acid and picric acid; and
(b) contacting a skin substrate with the soluble protein fraction.

8. The method according to claim 7, wherein the soluble protein fraction further comprises a protein inhibitor.

9. The method according to claim 7, wherein the soluble protein fraction is extracted from a leguminous seed.

10. The method according to claim 7, wherein the soluble protein fraction is extracted from a bambara nut seed.

11. A method of preventing skin aging, said method comprising:
(a) providing a soluble protein fraction, wherein the fraction (i) exhibits at least one band under non-reducing conditions in polyacrylamide gel electrophoresis with sodium dodecyl sulfate, (ii) has an average molecular weight of 500 to 500,000 dalton, (iii) has a total nitrogen content of from 0.005 to 0.5% by weight based on the percentage protein content and an amino nitrogen content of from 0.0005 to 0.01% by weight; and (iv) wherein the fraction is soluble in water and aqueous electrolyte solutions and insoluble in ethanol or acetone, and (v) wherein the fraction forms deposits in aqueous solution with a reactant selected from the group consisting of trichloroacetic acid and picric acid; and
(c) contacting a skin substrate with the soluble protein fraction.

12. The method according to claim 11, wherein the soluble protein fraction further comprises a protein inhibitor.

13. The method according to claim 11, wherein the soluble protein fraction is extracted from a leguminous seed.

14. The method according to claim 11, wherein the soluble protein fraction is extracted from a bambara nut seed.

15. A method of treating dry skin, said method comprising:
(a) providing a soluble protein fraction, wherein the fraction (i) exhibits at least one band under non-reducing conditions in polyacrylamide gel electrophoresis with sodium dodecyl sulfate, (ii) has an average molecular weight of 500 to 500,000 dalton, (iii) has a total nitrogen content of from 0.005 to 0.5% by weight based on the percentage protein content and an amino nitrogen content of from 0.0005 to 0.01% by weight; and (iv) wherein the fraction is soluble in water and aqueous electrolyte solutions and insoluble in ethanol or acetone, and (v) wherein the fraction forms deposits in aqueous solution with a reactant selected from the group consisting of trichloroacetic acid and picric acid; and
(d) contacting a dry skin substrate with the soluble protein fraction.

16. The method according to claim 15, wherein the soluble protein fraction further comprises a protein inhibitor.

17. The method according to claim 15, wherein the soluble protein fraction is extracted from a leguminous seed.

18. The method according to claim 15, wherein the soluble protein fraction is extracted from a bambara nut seed.
